(19) United States
(12) Patent Application Publication
(10) Pub. No.: US 2019/0000322 A1
(43) Pub. Date: Jan. 3, 2019

(54) PHOTOACOUSTIC PROBE AND PHOTOACOUSTIC APPARATUS INCLUDING THE SAME

(71) Applicant: CANON KABUSHIKI KAISHA, Tokyo (JP)

(72) Inventor: Naoto Abe, Machida-shi (JP)

(21) Appl. No.: 16/002,521

(22) Filed: Jun. 7, 2018

(30) Foreign Application Priority Data
Jun. 30, 2017 (JP) 2017-129350

(51) Int. Cl. A61B 5/00; G01N 29/24; G01N 29/28; G01N 29/44; A61B 5/145

(52) U.S. Cl. CPC A61B 5/0095; G01N 29/2418; A61B 5/14542; G01N 29/44; G01N 29/28

(57) ABSTRACT

A photoacoustic probe includes a light output portion that outputs light, an acoustic wave reception unit that receives a photoacoustic wave generated by irradiation of the light and outputs a reception signal, a transmission unit that transmits signal data based on the reception signal, a signal acquisition unit that converts the reception signal into a digital signal, and a memory unit that stores signal data based on the converted digital signal.

### BACKGROUND

### Field

The present disclosure relates to a photoacoustic probe and a photoacoustic apparatus including the probe. In particular, the present disclosure relates to a photoacoustic probe of a hand-held type that is applicable to wireless communication with a photoacoustic apparatus main body.

### Description of the Related Art

In recent years, as an imaging technique utilizing light, a photoacoustic apparatus that performs imaging of an inside of an object by utilizing a photoacoustic effect has been researched and developed. The photoacoustic apparatus performs reconstruction by using an ultrasonic wave (photoacoustic wave) generated by a photoacoustic effect from an optical absorber that absorbs energy of light radiated to the object and forms an absorption coefficient distribution image. Then, the apparatus generates a structural image or a functional image of the inside of the object from the absorption coefficient distribution image.

Similarly to an ultrasonic diagnosis apparatus, an apparatus including a shape of a hand-held probe enabling easy access to an observation part has also been researched and developed for photoacoustic apparatuses. Japanese Patent Laid-Open No. 2016-49215 discloses probes for photoacoustic imaging apparatuses, each of which includes an irradiator and a detector that detects a photoacoustic wave generated in a tested object and in which the irradiator is removably fitted to the detector. Japanese Patent Laid-Open No. 2016-49215 also describes that a memory storing light source information is provided in the photoacoustic probe and the light source information stored in the memory is transmitted to a controller in an apparatus main body in a wired or wireless manner. In addition to the controller, a reception circuit that amplifies a detection signal with respect to ultrasonic oscillating elements, an analog-to-digital converter that converts the detection signal amplified by the reception circuit into a digital signal, a data processor, an image merger, and the like are provided in the apparatus main body.

### SUMMARY

As a result of review of a technique described in Japanese Patent Laid-Open No. 2016-49215, the present inventor has confirmed that only light source information, such as a wavelength of an LED and a serial number, is stored in the memory inside the photoacoustic probe of Japanese Patent Laid-Open No. 2016-49215. The inventor arrived at an idea that, when photoacoustic data acquired by an acoustic probe can be transmitted to the apparatus main body in a wireless manner, convenience is enhanced without being affected by a cable connecting the probe and the apparatus main body, but when a communication state is not good due to a large amount of data, inconvenience such as loss of acquired signal data can occur. That is, in a case where wireless communication between the photoacoustic probe and the photoacoustic apparatus main body becomes unstable, data based on an acquired photoacoustic signal can be lost, which can result in degradation of image quality of an obtained photoacoustic image. A photoacoustic probe according to an aspect of the present disclosure includes a light output portion that outputs light, an acoustic wave reception unit that receives a photoacoustic wave generated by irradiation of the light and outputs a reception signal, a signal processor that converts the reception signal into a digital signal, a memory unit that stores signal data based on the digital signal, and a transmission unit that transmits the stored signal data.

Further features of the present disclosure will become apparent from the following description of exemplary embodiments with reference to the attached drawings.

### BRIEF DESCRIPTION OF THE DRAWINGS

FIG. 1 is a block diagram illustrating an example of a photoacoustic apparatus according to a first embodiment.

FIGS. 2A and 2B are schematic views of a probe of a hand-held type according to the first embodiment.

FIG. 3 is a block diagram illustrating an example of a computer and a peripheral configuration according to the first embodiment.

FIG. 4 is a timing view for explaining an operation of a photoacoustic probe in the first embodiment.

FIG. 5 is a timing view for explaining an operation of a photoacoustic apparatus main body in the first embodiment.

### DESCRIPTION OF THE EMBODIMENTS

Hereinafter, modes to implement the invention will be described with reference to drawings. Note that, dimensions, materials, shapes, and relative arrangement of components described below are to be appropriately changed based on a configuration of an apparatus to which the disclosure is applied and various conditions. Thus, the following description is not seen to be limiting. A photoacoustic probe includes a light output portion that outputs light, an acoustic wave reception unit that receives a photoacoustic wave generated by irradiation of the light and outputs a reception signal, and a transmission unit configured to transmit signal data based on the reception signal. The photoacoustic probe includes a signal processor that converts the reception signal into a digital signal and a memory unit that stores signal data based on the digital signal converted by the signal processor.

A photoacoustic apparatus includes the photoacoustic probe, a signal processor that creates image data based on the signal data obtained by the photoacoustic probe, and a display portion that displays an image based on the image data.

An object information acquisition method of the disclosure is an object information acquisition method of acquiring information of an inside of an object based on a signal caused by a photoacoustic wave generated by the object being irradiated with light. The method includes the steps of receiving the photoacoustic wave and outputting a reception signal, performing digital conversion of the reception signal, and storing signal data based on a signal subjected to the digital conversion, in which after the steps are performed in a photoacoustic probe, image data is created based on the stored signal data.

In the present specification, signal data based on a photoacoustic signal is data derived from a photoacoustic wave generated by light irradiation, and includes photoacoustic signal data obtained by converting photoacoustic signals output by a plurality of transducers into digital signals. The signal data based on the photoacoustic signal according to the disclosure is data obtained by processing only a photoacoustic signal from a target transducer and does not include signal data obtained by calculating photoacoustic signals of a plurality of transducers. In the present specification, an electrical signal refers to a concept including an analog signal and a digital signal unless otherwise described.

The disclosure relates to a technique of detecting an acoustic wave propagated from an object, storing data based on a detected photoacoustic signal, and generating and acquiring characteristic information of an inside of the object from the data based on the photoacoustic signal. The disclosure includes a photoacoustic probe, a photoacoustic apparatus including the probe, an object information acquisition method, and others. The disclosure also includes a method of acquiring a photoacoustic signal, a method of storing data based on the photoacoustic signal, and a signal processing method of the data based on the photoacoustic signal.

The disclosure includes a display method of generating and displaying an image indicating characteristic information of an inside of an object. The disclosure also includes a program that causes an information processing apparatus including a hardware resource, such as a CPU or a memory, to execute such methods, and a computer-readable non-transitory storage medium in which the program is stored.

A photoacoustic apparatus of the disclosure includes a photoacoustic imaging apparatus that utilizes a photoacoustic effect in which an acoustic wave generated in an inside of an object by irradiating the object with light (electromagnetic wave) is received and characteristic information of the object is acquired as image data. Here, the characteristic information is information of a characteristic value that corresponds to each of a plurality of positions inside the object and is generated by using a signal derived from the received photoacoustic wave.

In the present specification, photoacoustic image data refers to a concept including any image data derived from data based on a photoacoustic wave generated by light irradiation, that is, a photoacoustic signal. For example, the photoacoustic image data can be considered as image data representing at least one spatial distribution of object information regarding sound pressure (initial sound pressure) for generating a photoacoustic wave, an absorption energy density, an absorption coefficient, and a concentration (such as an oxygen saturation degree) of a substance forming the object.

Photoacoustic image data indicating spectral information, such as the concentration of the substance forming the object, is obtained based on photoacoustic waves generated by irradiation of light with a plurality of wavelengths that are different from each other. The photoacoustic image data indicating spectral information can be an oxygen saturation degree, a value obtained by weighting an oxygen saturation degree by magnitude of an absorption coefficient or the like, a total hemoglobin concentration, an oxyhemoglobin concentration, or a deoxyhemoglobin concentration. The photoacoustic image data indicating spectral information can be a glucose concentration, a collagen concentration, a melanin concentration, or volume fractions of fat and water.

Two-dimensional or three-dimensional characteristic information distribution is obtained based on characteristic information of positions inside the object. Distribution data can be generated as image data. The characteristic information can be obtained as distribution information of positions inside the object instead of as numerical value data. That is, distribution information, such as distribution of initial sound pressure, distribution of energy absorption density, distribution of an absorption coefficient, and distribution of an oxygen saturation degree, is obtained.

An acoustic wave in the present specification is typically an ultrasonic wave, and includes an elastic wave called a sound wave or an acoustic wave. An electrical signal converted from the acoustic wave by a transducer or the like is also called an acoustic signal. However, descriptions of an ultrasonic wave and an acoustic wave in the present specification are not intended to limit a wavelength of such elastic waves. An acoustic wave generated by a photoacoustic effect is called a photoacoustic wave or an optical ultrasonic wave. An electrical signal derived from a photoacoustic wave is also called a photoacoustic signal. Distribution data is called also photoacoustic image data or reconstruction image data.

In the following embodiments, a photoacoustic apparatus that irradiates an object with pulsed light, receives a photoacoustic wave from the object, and generates a blood vessel image (structural image) of an inside of the object will be described as an object information acquisition apparatus. Though a photoacoustic apparatus in which a photoacoustic apparatus main body and a hand-held photoacoustic probe exchange information through wireless communication mainly by an electric wave will be described in the following embodiments, the disclosure is also applicable to a photoacoustic apparatus that exchanges information, for example, through optical communication, electrical wiring, optical fiber, or the like instead of the wireless communication.

### First Embodiment

### (Apparatus Configuration)

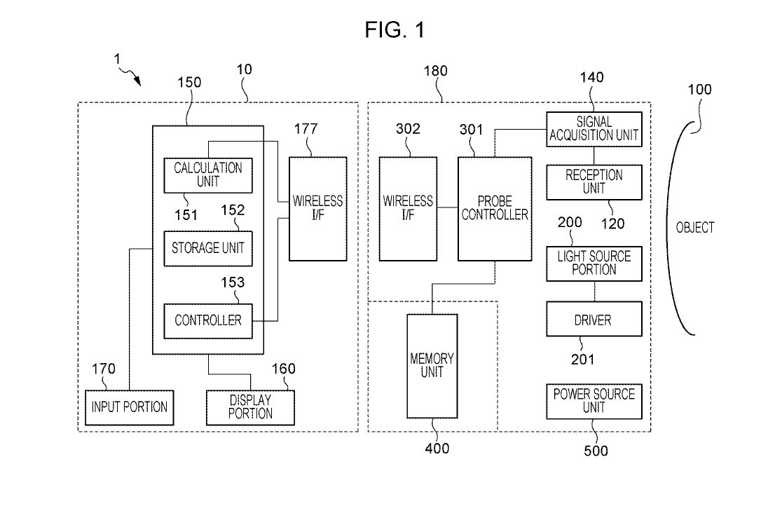

A photoacoustic apparatus 1 according to the present embodiment will be described below with reference to a block diagram of FIG. 1. The embodiment described here merely indicates an example of a mode to implement the disclosure and it is not always essential to include all parts described here to constitute a photoacoustic probe or a photoacoustic apparatus.

The photoacoustic apparatus 1 includes a photoacoustic apparatus main body 10 and a photoacoustic probe 180. The photoacoustic apparatus main body 10 includes a computer 150, a display portion 160, an input portion 170, and a wireless interface 177. The computer 150 includes a calculation unit 151, a storage unit 152, and a controller 153, and includes, for example, a function of creating image data based on signal data obtained in an ultrasonic probe.

The photoacoustic probe 180 includes a memory unit 400 and a signal processor 140 including a function of analog-to-digital conversion. The photoacoustic probe 180 also includes a light source portion 200, a driver 201, a reception unit 120, a probe controller 301, a wireless interface 302, and a power source unit 500. The signal processor 140 converts an analog signal, which is transmitted through the reception unit 120. The reception unit receives a photoacoustic wave that is generated internal to (including a surface) an object 100 when the object 100 receives light irradiation and outputs a reception signal as a digital signal so that digitization of the signal in the probe is performed and the signal is easily treated in the probe.

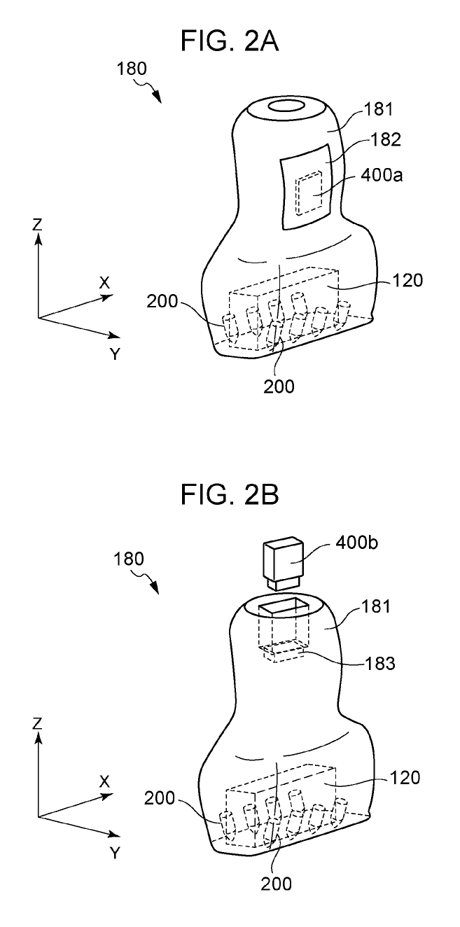

Including the memory unit 400 that stores signal data based on the digital signal converted by the signal processor 140 enables the data to be read out from the memory unit 400, even in, for example, a case where the signal data can be lost due to a poor communication state when the signal data is wirelessly transmitted to the photoacoustic apparatus main body 10. The photoacoustic probe 180 of the present embodiment will be described with reference to FIGS. 2A and 2B.

### (Photoacoustic Probe 180)

The photoacoustic probe 180 of the present embodiment will be described with reference to FIGS. 2A and 2B, which illustrate schematic views of the photoacoustic probe 180. As previously described, the photoacoustic probe 180 includes the light source portion 200, the driver 201, the reception unit 120, the signal processor 140, the probe controller 301, the wireless interface 302, memory units 40a (SD memory card) and 400b (USB memory), the power source unit 500, and a housing 181. In FIGS. 2A and 2B, the driver 201, the signal processor 140, the probe controller 301, and the wireless interface 302 can be omitted for ease of understanding. The housing 181 is a case that surrounds the light source portion 200, the driver 201, the reception unit 120, the signal processor 140, the probe controller 301, the wireless interface 302, and the memory unit 400. A user can utilize the photoacoustic probe 180 as a hand-held probe by holding the housing 181. Note that, “X”, “Y”, and “Z” axes in FIGS. 2A and 2B indicate coordinate axes when the probe stands still, but do not limit a direction of the probe when being used.

In the photoacoustic probe 180 illustrated in FIG. 2A, a cover 182 is openable and closable. FIG. 2A illustrates an example in which the SD memory card 400a is used as the memory unit 400. By opening the cover 182, the user can insert or remove the SD memory card 400a. An open/close portion of the cover 182 can include a waterproof construction so that water does not come into contact with the SD memory card 400a or a card slot (not illustrated) while inside the photoacoustic probe 180.

FIG. 2B illustrates an example of the photoacoustic probe 180 where the USB memory 400b is used as the memory unit 400. A USB connector 183 is provided. The USB memory 400b can be attached or detached by the user, and can include a waterproof structure. For example, the waterproof structure can be achieved by providing a cover (not illustrated) or providing a sealing material on a top part of a main body of the USB memory 400b.

### (Light Source Portion 200)

The light source portion 200 generates light to be radiated to the object 100. As the light source portion 200, a light source that generates pulsed light and outputs a plurality of wavelengths when acquiring substance concentration, such as an oxygen saturation degree, is desirable, for example. It is desired that the light source portion 200 is mounted in the housing 181 of the photoacoustic probe 180, and, in such a case, a semiconductor light emitting element, such as a semiconductor laser or a light emitting diode, is desirably used as illustrated in FIGS. 2A and 2B. The output of the plurality of wavelengths can be achieved by using a plurality of types of semiconductor lasers or light emitting diodes that generate light with different wavelengths and performing light emission in a switching manner.

A configuration can be such that a light source itself that constitutes the light source portion 200 is provided external to the photoacoustic probe 180, e.g., on the photoacoustic apparatus main body 10 side of the photoacoustic probe 180, as necessary to guide light into the photoacoustic probe 180 by using optical fiber or the like. A light output portion that outputs light refers to a light output end of the photoacoustic probe 180, which is provided in a region where the photoacoustic probe 180 is in contact with or proximate to the object 100.

Based on such a meaning, in a case where the light source portion 200 itself, as illustrated in FIG. 1, 2A, or 2B, is arranged at the end of the photoacoustic probe 180, the end itself of the light source serves as the light output portion. In a case where light is output from the photoacoustic probe 180 by guiding light from inside the photoacoustic probe 180 or outside the photoacoustic probe 180, the light output end of the photoacoustic probe 180 constitutes the light output portion. In the present embodiment, the semiconductor light emitting element is used as the light source of the light source portion 200.

A pulse width of the light emitted from the light source portion 200 is, for example, greater than or equal to 10 ns and less than or equal to 1 μs. While the wavelength of the light is desirably greater than or equal to 400 nm and less than or equal to 1600 nm, the wavelength can be decided based on light absorption characteristics of an optical absorber desired to be imaged.

In a case where, for example, a blood vessel is imaged at a high resolution, light with a wavelength (greater than or equal to 400 nm and less than or equal to 800 nm) that is largely absorbed by the blood vessel can be used. In a case where a portion of body more internal than a blood vessel is imaged, light with a wavelength (greater than or equal to 700 nm and less than or equal to 1100 nm) that is less absorbed at a background tissue (such as water or fat) of the body can be used.

When using a semiconductor light emitting element, a quantity of light may be insufficient to obtain a photoacoustic image. That is, a photoacoustic signal obtained by performing irradiation once does not always achieve a desired S/N ratio. In order to address this, light is emitted in a first cycle (light emission cycle), photoacoustic signals are added, and the resultant is averaged to enhance the S/N ratio. Based on the photoacoustic signal obtained through the addition average, photoacoustic image data is calculated in a second cycle (image frame rate cycle).

While the present embodiment is described using terms the first cycle (light emission cycle) and the second cycle (image frame rate cycle), a repetition interval of the “cycle” used in the present specification does not need to be completely constant. That is, in the present specification, in a case where the cycle is repeated with an inconstant time interval, the term of “cycle” is used. In particular, the first cycle also includes, for example, a cycle with a suspension period. A repetition time during a time not including a suspension period is called a cycle in the present specification.

An example of the wavelength of the light source portion 200 used in the present embodiment includes a wavelength of 797 nm. That is, the wavelength reaches a deep portion of an object and absorption coefficients of oxyhemoglobin and deoxyhemoglobin are substantially equal, so that such a wavelength is suitable for detection of a vascular structure. When a light source with the wavelength of 756 nm is used as a second wavelength, an oxygen saturation degree can be obtained by using a difference between the absorption coefficients of oxyhemoglobin and deoxyhemoglobin.

In the examples illustrated in FIGS. 2A and 2B, a plurality of (eight) semiconductor light emitting elements are arrayed as the light source portion 200 so that the light output end is formed at a tip of the housing 181. The number, arrangement, and the like of the light emitting elements are not limited thereto. For example, the number, arrangement, and the like are selected as appropriate based on a form or the like of a photoacoustic image to be obtained, e.g., based on whether to irradiate an object in a wide range.

### (Driver 201)

The driver 201, under control of the probe controller 301, drives, for example, the light source portion 200 to radiate pulsed light to the object 100 in the first cycle (light emission cycle). This results in a photoacoustic wave generated from the object 100 in the first cycle (light emission cycle). That is, based on an instruction of the probe controller 301, the driver 201 drives the light source portion 200 to emit light in the first cycle (light emission cycle). In a case where a plurality of light emitting diodes or semiconductor lasers are used as the light source portion 200, great light output is required to obtain a photoacoustic signal from the object 100. Large current needs to flow through the driver 201, thus, wiring of the driver 201 and the light source portion 200 is wired to minimize an inductance component.

(Reception unit 120)

The reception unit 120 receives the photoacoustic wave generated from the object 100 in the first cycle (light emission cycle) and outputs an electrical signal (photoacoustic signal) as an analog signal. The reception unit (acoustic wave reception unit) 120 receives the photoacoustic wave at a prescribed interval in the first cycle (light emission cycle). The reception unit 120 includes a transducer that receives the photoacoustic wave generated with the light emission in the first cycle (light emission cycle) and outputs the electrical signal, and a supporting member that supports the transducer. A piezoelectric material, a capacitive transducer (CMUT: Capacitive Micro-machined Ultrasonic Transducer), a transducer that utilizes a Fabry-Perot interferometer, or the like can be used as the transducer. Examples of the piezoelectric material include a piezoelectric ceramic material such as PZT (lead zirconate titanate) and a piezoelectric polymer film material such as PVDF (polyvinylidene fluoride).

The electrical signal obtained by the transducer in each first cycle (light emission cycle) is a time-resolved signal. An amplitude of the electrical signal indicates a value based on sound pressure received by the transducer at each time, e.g., a value proportional to the sound pressure.

A transducer that can detect a frequency component (typically, 100 KHz to 10 MHz) that forms the photoacoustic wave is desirable. It is also desired that the supporting member is configured in such a manner that a plurality of transducers are arrayed to form a flat surface or a curved surface as an array called a 1-dimensional array, a 1.5-dimensional array, a 1.75-dimensional array, or a 2-dimensional array.

It is desired that the transducer is arranged to completely surround the object 100 to detect an acoustic wave from various angles and enhance image accuracy. In a case where the object 100 is so large that the transducer cannot completely surround the object 100, the transducer can be arranged on a supporting member in a semispherical shape.

A medium that propagates the photoacoustic wave can be arranged in a space between the reception unit 120 and the object 100. This enables achieving matching of acoustic impedance at an interface between the object 100 and the transducer. Examples of the medium include water, oil, and ultrasonic gel.

In a case where the apparatus according to the present embodiment also generates an ultrasonic image by transmission and reception of the acoustic wave in addition to the photoacoustic image, the transducer can function as a transmission unit configured to transmit the acoustic wave. A transducer as a reception unit and a transducer as a transmission unit can be a single (shared) transducer or can be provided as different transducers.

### (Signal Processor 140)

The signal acquisition unit 140 (refer to FIG. 1) includes a function of converting an analog signal output from the reception unit 120 into a digital signal. For example, the signal acquisition unit 140 includes an amplifier that amplifies an electrical signal as an analog signal that is generated with light emission in each first cycle (light emission cycle) and output from the reception unit 120 and an analog-to-digital converter that converts the analog signal output from the amplifier into a digital signal. The amplifier can be configured to vary an amplification degree and the signal acquisition unit 140 can be constituted by an FPGA (Field Programmable Gate Array) chip or the like. Though illustrated as the signal acquisition unit 140 in FIG. 1, the signal acquisition unit 140 is to be considered as a signal processor in terms of the function.

An operation of the signal processor 140 will now be described. Analog signals output by a plurality of transducers of the reception unit 120 that are arranged in an array are amplified by a plurality of corresponding amplifiers and converted into digital signals by a plurality of corresponding analog-to-digital converters. The conversion is performed with an analog-to-digital conversion rate at least twice a band of a signal that is input. As described above, when the frequency component that forms the photoacoustic wave is 100 KHz to 10 MHz, the conversion is performed with a frequency of 20 MHz or more, desirably with a frequency of 40 MHz as the analog-to-digital conversion rate.

The signal processor 140 synchronizes timing of light irradiation with timing of signal collection processing by using a light emission control signal. That is, the analog-to-digital conversion is started with the analog-to-digital conversion rate described above with a light emission time as a reference in each first cycle (light emission cycle) and the analog signals are converted into the digital signals. As a result, a digital data line in each time interval (analog-to-digital conversion interval) corresponding to 1/analog-to-digital conversion rate from the light emission time in each first cycle (light emission cycle) can be obtained in each of the plurality of transducers. The signal processor 140 is also called a Data Acquisition System (DAS).

### (Probe Controller 301)

The probe controller 301 (refer to FIG. 1) controls light emission timing of the light source portion 200, an analog-to-digital conversion rate, analog-to-digital conversion timing, or the like, and acquires photoacoustic data of each light emission of the light source portion 200. The probe controller 301 transmits the photoacoustic data to the computer 150 via the wireless interface 302 and the wireless interface 177. At the same time, the probe controller 301 writes, in the memory unit 400, the photoacoustic data acquired in each light emission of the light source portion 200. That is, the probe controller 301 also includes a function of a memory controller. The probe controller 301 performs control to store the signal data in the memory unit 400 based on the digital signal to be obtained for each of the plurality of transducers as described in the reception unit 120.

### (Wireless Interfaces 177, 302)

The wireless interface 177 and the wireless interface 302 are wireless interfaces that perform bidirectional communication. A wireless interface that performs data communication conforming to wireless LAN standard, such as Wi-Fi®, is suitably used. At least photoacoustic data acquired in the first cycle (light emission cycle) can have a communication speed at which the photoacoustic data can be transmitted to the photoacoustic apparatus main body 10 within the first cycle (light emission cycle). Setting data or the like from the photoacoustic apparatus main body 10 can also be transmitted to the photoacoustic probe 180 via the wireless interface 177 and the wireless interface 302. It is also possible that a wireless interface, that is, a wireless communication unit receives control data from the photoacoustic apparatus main body 10, and the probe controller 301 controls an operation of the photoacoustic probe 180 based on the control data.

### (Memory Unit 400)

It is desired that the memory unit 400 be an attachable/detachable non-volatile memory, for example, a flash memory. In a case where a non-volatile memory, such as a flash memory, is used, photoacoustic data can be read by the photoacoustic apparatus main body 10 or another computer by detaching the non-volatile memory from the photoacoustic probe 180 after measurement. During an operation in which the photoacoustic data is read by another computer after detaching the memory from the photoacoustic probe 180, a different memory (flash memory) is mounted in the photoacoustic probe 180 so that measurement and reading can be performed at the same time. Use of a USB memory, an SD memory card, or the like with high versatility as the flash memory brings an advantage that a new special reading device is not required when photoacoustic data is read by the photoacoustic apparatus main body 10 or another computer.

While the memory unit 400 is described as an attachable/detachable non-volatile memory in the present embodiment, the memory unit 400 can be a volatile memory, such as a RAM. In a case where a volatile memory, such as a RAM, is used, the memory unit 400 is always supplied with power so that stored photoacoustic data is not lost. The photoacoustic data can be transmitted to the photoacoustic apparatus main body 10 or another computer through an interface (not illustrated) conforming to USB standard or IEEE1394 standard, or the like, the wireless interface 177, and the wireless interface 302 after measurement.

A unit configured to preform transmission of the photoacoustic data stored in the memory unit 400, such as transmission of the photoacoustic data by attachment/detachment of a non-volatile memory and transmission of the photoacoustic data through an interface conforming to serial bus standard such as USB standard or IEEE1394 standard is called a data transmission unit in the present specification. In a case where data stored in the memory unit 400 is read, the read data is deleted by the probe controller 301, thus generating an unused storage region in the memory unit 400.

### (Power Source Unit 500)

The power source unit 500 is mounted inside the housing 181 and supplies electric power to the light source portion 200, the driver 201, the reception unit 120, the signal processor 140, the probe controller 301, the wireless interface 302, and the memory unit 400 that are inside the photoacoustic probe 180. Since large electric power is needed, particularly for light emission of the light source, a secondary battery, such as a nickel hydride battery, a lithium ion battery, or a lithium polymer battery, with high energy density is used as the power source unit 500. Use of a rechargeable battery enables, for example, saving time and effort of replacing the battery. While a secondary battery is used as an example of the power source, the power source unit 500 can, for example, be connected to the photoacoustic apparatus main body 10 using a power source cable (not illustrated) to supply electric power. In such a case, when the power source cable is connected between the photoacoustic apparatus main body 10 and the photoacoustic probe 180, a degree of freedom of movement can be reduced. However, this arrangement can prevent occurrence of electric power not being supplied because of discharge of the battery.

Next, a constituent part of the photoacoustic apparatus main body 10 will be described.

### (Computer 150)

The computer 150 includes the calculation unit 151, the storage unit 152, and the controller 153, and combines pieces of photoacoustic data, output from the wireless interface 177 in each first cycle (light emission cycle), based on the second cycle (hereinafter, also referred to as an image frame rate cycle) and stores the resultant in the storage unit 152 as data based on the photoacoustic signal. Combining the photoacoustic data includes simple addition, as well as weighting addition, addition average, moving average, and the like. While the following description will mainly discuss, as an example, taking the addition average, a combining method other than the addition average is also applicable. The computer 150 performs processing, such as image reconstruction, for signal data based on the photoacoustic signal, which is stored in the storage unit 152, and thereby generates photoacoustic image data within a prescribed period in the second cycle (image frame rate cycle). The computer 150 can be considered as an image signal processor.

The display portion 160 displays an image based on the photoacoustic image data of the second cycle (image frame rate cycle). In a case of a frame rate by which the display portion 160 is not able to display the photoacoustic image data of the second cycle (image frame rate cycle), a frame rate converter 159 can convert the photoacoustic image data, which is generated in the second cycle (image frame rate cycle), to have a frame rate suitable for display on the display portion 160.

The computer 150 can perform, as necessary, image processing for display with respect to the obtained photoacoustic image data or processing for composing graphic for GUI with the obtained photoacoustic image data.

The calculation unit 151 includes a calculation function and can include a processor, such as a CPU or a GPU (Graphics Processing Unit), or a calculation circuit, such as an FPGA (Field Programmable Gate Array) chip. The calculation unit 151 can include a single processor or a calculation circuit or can include a plurality of processors or calculation circuits.

The computer 150 adds data of the same time based on the light emission time in a digital data line output from the wireless interface 177 in each first cycle (light emission cycle) and averages the resultant. A digital signal subjected to the addition average is stored in the storage unit 152 as photoacoustic data after the addition average in each second cycle (image frame rate cycle).

Based on the photoacoustic data subjected to the addition average and stored in the storage unit 152, the calculation unit 151 executes generation of photoacoustic image data (structural image or functional image) by image reconstruction and other various calculation processing in each second cycle (image frame rate cycle). The calculation unit 151 can receive, from the input portion 170, various parameter inputs, such as a measurement condition, an object sound speed, and a configuration of a holding portion, and use such parameters for calculation.

Any method, such as a back projection method in time domain, a back projection method in Fourier domain, and a model-based method (repetitive calculation method), can be adopted as a reconstruction algorithm used for the calculation unit 151 to convert an electrical signal into three-dimensional volume data. Examples of the back projection method in time domain include Universal Back-Projection (UBP), Filtered Back-Projection (FBP), or Delay-and-Sum.

In a case where the light source portion 200 can output light by switching two wavelengths, the calculation unit 151 can generate, through image reconstruction processing, first initial sound pressure distribution from a photoacoustic signal derived from light with a first wavelength and second initial sound pressure distribution from a photoacoustic signal derived from light with a second wavelength. The calculation unit 151 acquires first absorption coefficient distribution by correcting the first initial sound pressure distribution by light quantity distribution of the light with the first wavelength and acquires second absorption coefficient distribution by correcting the second initial sound pressure distribution by light quantity distribution of the light with the second wavelength. The calculation unit 151 can acquire oxygen saturation distribution from the first and second absorption coefficient distribution. Details and order of the calculation are not limited thereto as long as the oxygen saturation distribution can be finally obtained.

The storage unit 152 includes a volatile memory, such as a Random Access Memory (RAM) or a non-transitory storage medium, such as a Read Only Memory (ROM), a magnetic disk, or a flash memory. A storage medium in which a program is stored is a non-transitory storage medium. The storage unit 152 can include a plurality of storage media.

The storage unit 152 can save various data, such as photoacoustic data subjected to addition average in the second cycle (image frame rate cycle), photoacoustic image data generated by the calculation unit 151, or reconstruction image data based on photoacoustic image data.

The controller 153 includes a calculation element, such as a CPU. The controller 153 controls an operation of each of the components of the photoacoustic apparatus 1. The controller 153 can receive an instruction signal based on various operations, such as a start of measurement, from the input portion 170, and control each of the components of the photoacoustic apparatus 1.

The controller 153 reads out a program code stored in the storage unit 152, and controls an operation of each of the components of the photoacoustic apparatus 1. The controller 153 transmits, to the probe controller 301 via the wireless interface 177 and the wireless interface 302, an instruction from a user or a setting value (such as a first cycle (light emission cycle), the number of times of repetition, a light quantity of a light source, or an analog-to-digital conversion rate), from the photoacoustic apparatus main body 10.

The computer 150 can be a specifically designed workstation. The computer 150 can be a versatile PC or workstation operating based on an instruction of a program stored in the storage unit 152. Each of the components of the computer 150 can be different hardware. Alternatively, at least a part of the components of the computer 150 can be the same hardware.

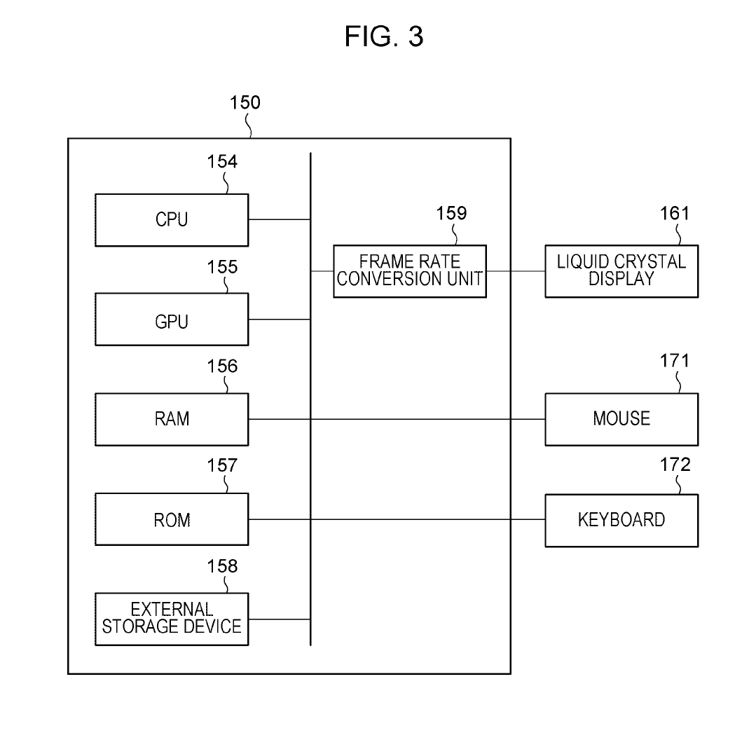

FIG. 3 illustrates a specific configuration example of the computer 150 according to the present embodiment. The computer 150 according to the present embodiment includes a CPU 154, a GPU 155, a RAM 156, a ROM 157, and an external storage device 158. A liquid crystal display 161 as the display portion 160, a mouse 171 and a keyboard 172 as the input portion 170 are connected to the computer 150.

The computer 150 and the reception unit 120 can be provided with a configuration to be housed in a common case. A computer housed in the case can perform a part of signal processing and a computer provided outside the case can perform the other signal processing. In such a case, the computers provided inside and outside the case can collectively be considered as the computer according to the present embodiment. That is, the hardware constituting the computer may not be housed in one case. An information processing apparatus that is installed in a remote place and provided by a cloud computing service or the like can be used as the computer 150.

### (Display Portion 160)

The display portion 160 can include a display, such as a liquid crystal display or an organic EL (Electro Luminescence) display. The display portion 160 is a device that displays an image based on object information or the like, a numerical value at a specific position or the like, etc., that are acquired by the computer 150. The display portion 160 displays the aforementioned reconstruction image data with a frame rate (image frame rate) of the second cycle or reconstruction image data whose frame rate is converted by a frame rate converter (not illustrated). The frame rate of the display portion 160 is, for example, generally 50 Hz, 60 Hz, 72 Hz, or 120 Hz. By adjusting the frame rate (image frame rate) of the second cycle to the frame rate of the display portion 160, a configuration in which the frame rate converter (not illustrated) is not necessary can be realized. The display portion 160 can display a GUI for operating an image or an apparatus. The display portion 160 or the computer 150 can perform image processing, such as adjustment of a luminance value.

A user, such as a physician or technician, checks a photoacoustic image displayed on the display portion 160. The image displayed on the display portion 160 can be saved, for example, in a memory in the computer 150 or a data management system connected to the photoacoustic apparatus 1 via a communication network, based on a saving instruction from the user or the computer 150. The display portion 160 can display a GUI or the like in addition to an image generated by the computer 150. The display portion 160 can display a charged state of the power source unit 500 of the photoacoustic probe 180 or a use state of the memory unit 400.

### (Input Portion 170)

The input portion 170 enables the user to input information and includes a function of receiving an instruction of the user or the like. Using the input portion 170, the user can, for example, perform an operation of an instruction to start or end measurement or an instruction to save a created image. A mouse, a keyboard, a dedicated knob, etc. can be used as the input portion 170 enabling a user operation.

The display portion 160 can be a touch panel so that the display portion 160 is utilized as the input portion 170. The input portion 170 receives an instruction from the user, an input of a numerical value, or the like and transmits the instruction or the input to the computer 150. As described above, the input portion 170 also includes an interface of USB standard, IEEE1394 standard, or the like or an interface such as an SD memory card reader in order to read content of the memory unit 400 of the photoacoustic probe 180.

The components of the photoacoustic apparatus 1 can be different devices or an integrated device. At least a part of the components of the photoacoustic apparatus 1 can be an integrated device. The photoacoustic apparatus 1 can include a holding member that holds the object 100 and stabilizes a shape of the object 100. A member in which both optical transparency and acoustic wave transparency are high is desirable as the holding member. For example, polymethylpentene, polyethylene terephthalate, acryl, or the like can be used.

### (Object 100)

While the object 100 is not part of the photoacoustic apparatus 1 of the present application, it will be described below. The photoacoustic apparatus 1 according to the present embodiment can be used for the purpose of a diagnosis, a follow-up of a chemical treatment of a malignant tumor, a vascular disease, or the like, of a human or an animal. Therefore, a site targeted for the diagnosis, such as a breast, an organ, a vasoganglion, a head, a neck, an abdomen, or limbs including fingers and/or toes of a human or an animal is expected as the object 100. For example, in a case where a measurement target is a human body, oxyhemoglobin, deoxyhemoglobin, a blood vessel including a large amount of them, a new blood vessel formed near a tumor, or the like can be considered as a target of an optical absorber. Plaque of a carotid artery wall or the like can be a target of an optical absorber. A pigment, such as methylene blue (MB) or indocyanine green (ICG), fine gold particles, or an externally introduced substance integrating or chemically modifying them, can be used as an optical absorber. A puncture needle or an optical absorber attached to a puncture needle can be used as an object to be observed. The object 100 can be an inanimate object, such as a phantom or a test object.

### (Explanation for Operation)

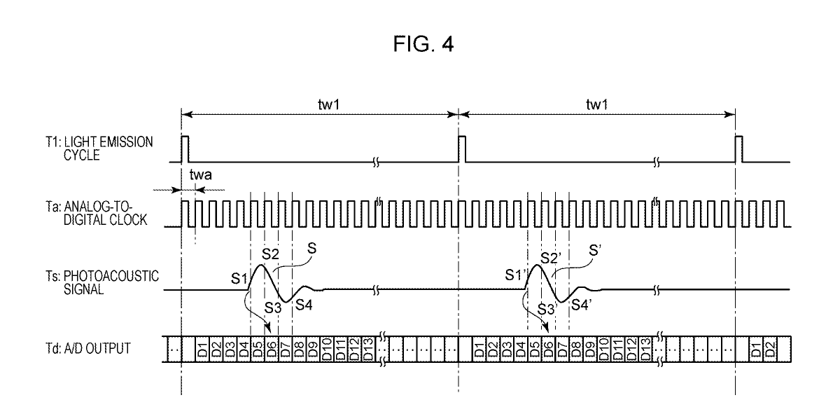

FIG. 4 is a timing view for explaining an operation of the photoacoustic probe 180 in the first embodiment. In FIG. 4, a horizontal axis is a time axis.

The controller 153 transmits an instruction from the user or a setting value from the photoacoustic apparatus main body 10 through the wireless interface 177 and the wireless interface 302 to the probe controller 301. Then, the probe controller 301 performs the following control based on the setting value. The probe controller 301 includes a microcomputer, and can be relatively easily realized when control is performed by firmware. The probe controller 301 can be an FPGA or dedicated hardware instead of a microcomputer.

In FIG. 4, a cycle of T1 indicates the first cycle (light emission cycle: tw1). A light emission control signal is denoted by T1. The light source portion 200 of the photoacoustic probe 180 emits light at a rising edge of T1, and a photoacoustic signal associated with the light emission is acquired as follows in each analog-to-digital clock (analog-to-digital conversion rate: twa), where an analog-to-digital clock is denoted by Ta.

The analog-to-digital converter converts a photoacoustic signal Ts that is an analog signal into a digital signal at a rising edge of the analog-to-digital clock. As illustrated by Td in FIG. 4, the analog-to-digital converter outputs photoacoustic data (D1, D2, D3, . . . ), which is converted into a digital signal, based on the light emission control signal (light emission of the light source). The light source emits light in response to a light emission control signal in a next cycle, and the analog-to-digital converter outputs photoacoustic data (D1′, D2′, D3′, . . . ), which is converted into a digital signal, based on the light emission control signal (light emission of the light source). The probe controller 301 stores the obtained photoacoustic data in the memory unit 400 and outputs the obtained photoacoustic data to the computer 150 via the wireless interface 177 and the wireless interface 302.

A length of the first cycle (light emission cycle: tw1) can be set in consideration of a maximum permissible exposure (MPE) against a skin. This is because a value of the MPE is reduced as the length of the first cycle tw1 is shorter. For example, in a case where a measurement wavelength is 750 nm, a pulse width of pulsed light is 1 μsec, and the first cycle tw1 is 0.1 msec, the value of the MPE against the skin is about 14 J/m².

In a case where peak power of pulsed light radiated from a light output portion 113 is 2 kW and an irradiation area from the light output portion 113 is 150 mm², light energy radiated to the object 100, such as a human body, from the light source portion 200, is approximately 13.3 J/m². In such a case, light energy radiated from the light output portion 113 is less than or equal to the value of the MPE. In this manner, when the first cycle tw1 is greater than or equal to 0.1 msec, it is possible to ensure that the light energy is less than or equal to the value of the MPE.

As described above, the light energy is set in a range not exceeding the value of the MPE based on the first cycle tw1, the peak power of the pulsed light, and the irradiation area. In a case where a semiconductor light emitting element is used as the light source, a photoacoustic wave generated internal to the object is extremely faint. Thus, in order to improve the S/N ratio of the photoacoustic signal, the photoacoustic apparatus 1 adds digital signals of the same time in based on the light emission control signal and averages the resultant.

In order to improve the S/N ratio of the photoacoustic signal, the number of times of addition average needs to be increased as described below, but the number of times of addition average is set as twice in FIG. 4 for ease of understanding. That is, a timing view in which photoacoustic signals associated with light emission occurring twice are added and the resultant is averaged to improve the S/N ratio is used for explanation. Specifically, in Td of FIG. 4, pieces of photoacoustic data (D1 and D1′, D2 and D2′, D3 and D3′, . . . ) given the same number are subjected to addition average. Through the addition average, the S/N ratio of the photoacoustic data can be improved. The first embodiment has a configuration in which the photoacoustic apparatus main body 10 performs such addition average.

The photoacoustic data indicated in Td of FIG. 4 described above is transmitted to the computer 150 of the photoacoustic apparatus main body 10 through the wireless interface 302 and the wireless interface 177. The computer 150 performs the addition average processing described above. The computer 150 performs the following processing and generates reconstruction image data.

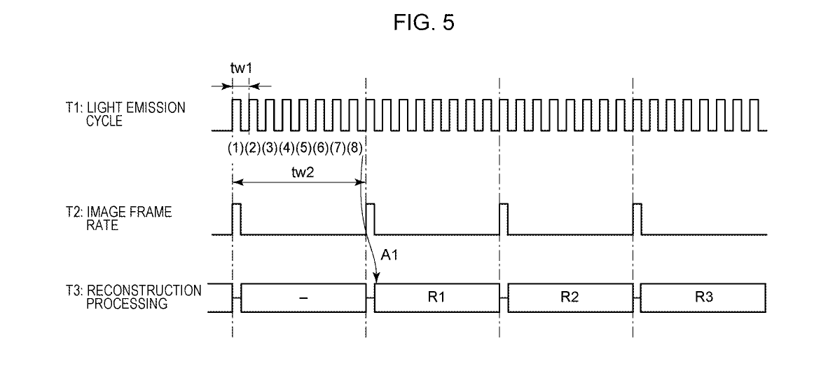

An operation of the photoacoustic apparatus main body 10 will be described with reference to FIG. 5. FIG. 5 is a timing view for explaining an operation of the photoacoustic apparatus main body 10 in the first embodiment of the invention. In FIG. 5, a horizontal axis is a time axis.

As illustrated from T1 to T3 in FIG. 5, the light source portion 200 irradiates an object with light eight times in the first cycle: tw1, and photoacoustic signals generated from the object in association with each light irradiation are obtained ((1) to (8)). The number of times of light emission is set as eight times for ease of illustration of the timing view of FIG. 5, but the number is not limited thereto.

The computer 150 adds the obtained photoacoustic data and averages the resultant, and obtains photoacoustic data A1 subjected to the addition average in each image frame rate cycle: tw2. Simple average, moving average, weighted average, or the like can be performed instead of the addition average. As a specific example of a numerical value, in a case where a time of the first cycle tw1 is 0.1 msec and the image frame rate is 60 Hz, the image frame rate cycle: tw2 is 16.7 msec, so that the number of times of addition average in the image frame rate cycle can be set as 167 times.

By performing processing for reconstruction based on the photoacoustic data A1 subjected to the addition average during a prescribed time interval in the second cycle (image frame rate cycle) tw2, reconstruction image data R1 is obtained. Reconstruction image data is sequentially calculated by the calculation unit 151 in the image frame rate cycle tw2 and the reconstruction image data is sequentially displayed on the display portion 160. The user can make a diagnosis or the like by observing the display portion 160.

In a case where photoacoustic data is transmitted via a wireless interface, however, an image can be disturbed, for example, due to exogenous noise or interference with another wireless device. According to the first embodiment, a configuration is such that photoacoustic data transmitted to the photoacoustic apparatus main body 10 is stored in the memory unit 400 in the photoacoustic probe 180. Thus, even when communication is unstable, for example, the photoacoustic data stored in the memory unit 400 is read by the photoacoustic apparatus main body 10 or another computer after measurement and addition average and reconstruction processing are performed, thus making it possible to obtain a reconstruction image without image disorder.

In a case where the photoacoustic data stored in the memory unit 400 is read by the photoacoustic apparatus main body 10 or another computer after measurement, the data transmission unit described above can be used. Alternatively, using the probe controller 301, signal data based on a digital signal (obtained by the signal processor) that has a data amount less than or equal to a data amount stored in the memory unit 400 can be output by the wireless communication unit.

In a case where an interface conforming to serial bus standard, such as USB standard or IEEE1394 standard, is used as the data transmission unit, for example, charging of a second battery of the power source unit 500 can be simultaneously performed. In a case where the wireless interface 177 and the wireless interface 302 are used as the data transmission unit, for example, a probe holder can be provided. The probe holder is installed in a location where a communication state of the wireless interface is good. By providing, in the probe holder, an electrode and a connector for performing charging of the second battery of the power source unit 500 of the photoacoustic probe 180 and setting the photoacoustic probe 180 in the probe holder, charging can be started and photoacoustic data can be automatically read by the photoacoustic apparatus main body 10 or another computer at the same time.

According to the present embodiment, even in a case where communication from the photoacoustic probe 180 to the photoacoustic apparatus main body 10 is unstable, the photoacoustic data stored in the memory unit 400 is read by the photoacoustic apparatus main body 10 or another computer after measurement and addition average and reconstruction processing are performed so that a reconstruction image without image disorder can be obtained. As a result, the user can confirm the reconstruction image without image disorder after measurement even when the communication state of the wireless interface is bad.

### Second Embodiment

A second embodiment provides a mode in which communication capacity of the wireless interface is further reduced. By further reducing the communication capacity, reliability of the wireless interface can be increased. For example, it is possible to perform processing of addition, retransmission, or the like of data for performing processing such as error correction.

In the second embodiment, signal data based on a photoacoustic signal is further compressed and transmitted. For example, photoacoustic signals that are output from the respective transducers have a high correlation in a direction of the time axis. By utilizing such a relation and performing, for example, Differential Pulse Code Modulation (DPCM) or the like to compress the signal data, new signal data can be generated and transmitted through the wireless interface 302 and the wireless interface 177. The DPCM or the like can be performed for pieces of data of the same time transmitted in each first cycle (light emission cycle) to perform compression and transmission. Alternatively, irreversible compression can be performed. The DPCM or the like can be performed for pieces of data of the same time transmitted in each second cycle (image frame rate cycle) to perform compression and transmission. Alternatively, irreversible compression can be performed. Data transmitted in each second cycle (image frame rate cycle) can be subjected to thinning-out (image frame thinning-out) and data based on the photoacoustic signal can be transmitted.

In a case where the irreversible compression is performed, the data based on the photoacoustic signal, which is transmitted via the wireless interface, is inaccurate data, and degradation of a reconstruction image is caused. Even when the image frame thinning-out is performed, degradation of the reconstruction image, in which a motion of the reconstruction image is not smooth, is caused.

In a case where such degradation of image quality is caused, when the user needs to view an accurate reconstruction image, the user can use the transmission unit to read signal data based on a photoacoustic signal, which is stored in the memory unit 400 in the photoacoustic probe 180, and create and observe reconstruction data. In this manner, according to the second embodiment, it is possible to further reduce the communication capacity of the wireless interface, thus making it possible to increase reliability of the wireless interface.

Similarly to the first embodiment, even when the communication state from the photoacoustic probe 180 to the photoacoustic apparatus main body 10 is unstable, a reconstruction image without image disorder can be obtained after measurement from data based on the photoacoustic signal, which is stored in the memory unit 400. In a case where irreversible compression or image frame thinning-out is performed, the user cannot view an accurate image at the time of measurement, but can obtain an accurate reconstruction image after the measurement from data based on the photoacoustic signal, which is stored in the memory unit 400.

As a result, the user can confirm the accurate reconstruction image without image disorder as necessary.

### Third Embodiment

Any data transmission unit described above can prevent insufficiency of memory capacity by transmitting (reading) data based on a photoacoustic signal, which is stored in the memory unit 400, and then deleting the read data based on the photoacoustic signal. It can take time and effort for the user to manage transmission of signal data based on the photoacoustic signal and delete the signal data, and there is possibility that the user erroneously deletes other data based on a photoacoustic signal. In order to address this, transmission flag data can be added to each of signal data based on the photoacoustic signal and a transmission flag can be set when the signal data is read. For example, in a case where a transmission flag is set, the probe controller 301 can delete the corresponding signal data based on the photoacoustic signal.

In a case where available capacity of the memory unit 400 is less than a given value, for example, the display portion 160, serving as a warning unit, of the photoacoustic apparatus main body 10 can display a message such as “memory capacity is insufficient” or a sound output portion (not illustrated), also serving as a warning unit, can generate a warning sound or the like.

In such a case, the probe controller 301 transmits the available capacity of the memory unit 400 to the photoacoustic apparatus main body 10 via the wireless interface 302 and the wireless interface 177. The computer 150 of the photoacoustic apparatus main body 10 can calculate a measurable time from the available capacity of the memory unit 400 and display the “measurable time”, for example, on the display portion 160 of the photoacoustic apparatus main body 10. While not illustrated, a device (warning unit) that provides display, light emission, or sound can be mounted in the photoacoustic probe 180 itself so that a message is displayed or a warning sound or the like is generated.

In a case where the available capacity of the memory unit 400 becomes insufficient during measurement, the probe controller 301 can delete data based on the photoacoustic signal, which is stored in the memory unit 400, in order beginning from older data so that the available capacity is ensured.

In a case where power source capacity of the power source unit 500 is a less than or equal to a given value, a measurable time is limited. Then, similarly to the case where the capacity of the memory unit 400 is insufficient, the probe controller 301 transmits the power source capacity of the power source unit 500 to the photoacoustic apparatus main body 10 via the wireless interface 302 and the wireless interface 177.

The display portion 160 of the photoacoustic apparatus main body 10 then, for example, can display a message such as “power source capacity is insufficient” or a sound output portion (not illustrated) can generate a warning sound or the like. The computer 150 of the photoacoustic apparatus main body 10 can calculate a measurable time from the power source capacity of the power source unit 500 and display the “measurable time”, for example, on the display portion 160 of the photoacoustic apparatus main body 10. A device that provides display, light emission, or sound can be mounted in the photoacoustic probe 180 itself so that a message is displayed or a warning sound or the like is generated.

As described above, by managing the available capacity of the memory unit 400 or the power source capacity of the power source unit 500, it is possible to prevent the capacity of the memory unit 400 or the power source capacity of the power source unit 500 from becoming insufficient during measurement and not enabling measurement to be performed.

### Other Embodiments

The probe controller 301 stores, in the memory unit 400, at least one or more information of an ID specifying a patient, a measurement condition, a measurement time, a user name, and a hospital name with signal data based on a photoacoustic signal. The computer 150 of the photoacoustic apparatus main body 10 transmits such information to the probe controller 301 via the wireless interface 177 and the wireless interface 302 before measurement.

By storing an ID specifying a patient, a measurement condition, a measurement time, a user name, a hospital name, or the like with the data based on the photoacoustic signal in the memory unit 400 as described above, when the data based on the photoacoustic signal is transmitted to the photoacoustic apparatus main body 10 or another computer after measurement, information of the data based on the photoacoustic signal at the time of measurement can easily be managed. While the configuration in which the wireless interface 177 and the wireless interface 302 are used for wireless communication by the electric wave is indicated as an example, wireless optical communication, communication through optical fiber, or wired communication is also applicable.

A photoacoustic probe of the disclosure includes a signal processor that converts a reception signal based on a photoacoustic wave into a digital signal, and a memory unit that stores signal data based on the digital signal converted by the signal processor. Thus, even when communication between the photoacoustic probe and a photoacoustic apparatus main body is unstable, it is possible to confirm a reconstruction image by reading the signal data based on the photoacoustic signal from the memory unit after measurement.

### Additional Embodiments

Embodiment(s) can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While exemplary embodiments have been described, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2017-129350 filed Jun. 30, 2017, which is hereby incorporated by reference herein in its entirety.

What is claimed is:

1. A photoacoustic probe comprising:
a light output portion that outputs light;
an acoustic wave reception unit that receives a photoacoustic wave generated by irradiation of the light and outputs a reception signal;
a signal processor that converts the reception signal into a digital signal;
a memory unit that stores signal data based on the digital signal; and
a transmission unit that transmits the stored signal data.

2. The photoacoustic probe according to claim 1, wherein the transmission unit is a wireless communication unit configured to wirelessly transmit the stored signal data.

3. The photoacoustic probe according to claim 1, wherein the transmission unit uses an interface conforming to a serial bus standard.

4. The photoacoustic probe according to claim 1, wherein the memory unit is a memory that is attachable to or detachable from the photoacoustic probe.

5. The photoacoustic probe according to claim 5, wherein the memory is a non-volatile memory.

6. The photoacoustic probe according to claim 1, further comprising a power source unit.

7. The photoacoustic probe according to claim 6, wherein power source capacity of the power source unit or available capacity of the memory unit is wirelessly transmitted to the photoacoustic apparatus main body via the transmission unit.

8. The photoacoustic probe according to claim 1, further comprising a probe controller that controls storing the signal data in the memory unit.

9. The photoacoustic probe according to claim 8, wherein the probe controller controls storing the signal data in the memory unit based on the digital signal obtained for each of a plurality of transducers.

10. The photoacoustic probe according to claim 8, wherein the probe controller causes the transmission unit to wirelessly output the stored signal data with a data amount less than or equal to a data capacity of the memory unit.

11. The photoacoustic probe according to claim 8, wherein the transmission unit further receives control data from a photoacoustic apparatus main body, and
wherein the probe controller controls an operation of the photoacoustic probe based on the control data.

12. The photoacoustic probe according to claim 8, wherein the probe controller controls storing one or more of information of an ID specifying a patient, a measurement condition, a measurement time, a user name, and a hospital name with the stored signal data based on the digital signal in the memory unit.

13. The photoacoustic probe according to claim 8, wherein after the transmission unit transmits the stored signal data, the probe controller deletes the transmitted data from the memory unit.

14. The photoacoustic probe according to claim 8, further comprising a power source unit, wherein in a case where power source capacity of the power source unit or available capacity of the memory unit is less than a given value, the probe controller controls a warning unit to provide a warning.

15. The photoacoustic probe according to claim 14, wherein the warning unit one or more of a display portion and a sound output portion that are mounted in the photoacoustic probe.

16. A photoacoustic apparatus comprising:
the photoacoustic probe according to claim 1;
an image signal processor that creates image data based on the signal data obtained by the photoacoustic probe; and
a display portion that displays an image based on the image data.

17. An object information acquisition method for acquiring information of an object, the method comprising:
generating a photoacoustic wave;
receiving the photoacoustic wave;
outputting a reception signal based on the received photoacoustic wave;
performing digital conversion of the reception signal;
storing signal data based on a signal subjected to the digital conversion; and
creating image data based on the stored signal data.
